(19) United States
(12) Patent Application Publication
(10) Pub. No.: US 2023/0226243 A1
(43) Pub. Date: Jul. 20, 2023

(54) SANI- FRESH

(71) Applicant: Tanzy Shy, Famington Hills, MI (US)

(72) Inventor: Tanzy Shy, Famington Hills, MI (US)

(21) Appl. No.: 17/578,626

(22) Filed: Jan. 19, 2022

(51) Int. Cl. A61L 9/12

(52) U.S. Cl. CPC A61L 9/12

(57) ABSTRACT

The invention relates to enhancing any area that has a blowing air Vent. By dispensing a sanitized aromatic solution that will sanitize any Area. It will eliminate viruses or other airborne contaminants. The Specification outlines how the product will improve and sanitize The air quality of the customers surroundings.

The ingredients to formulate the sanitize solution to ensure that the Health, safety and environmental requirements are achieved if not Exceeded.
Can last up to 30 days
Built in battery gauge
Dispenser—compact for any area that has an air vent

The product claim is safe to use in all environments and is not Harmful to children, pets or plants. To validate the claim the product Is evaluated in various settings to confirm the performance meets the user's satisfaction. As there are similar products on the market, any potential risk has been mitigated and there will be no underlying affects.

### SPECIFICATION/DESCRIPTION

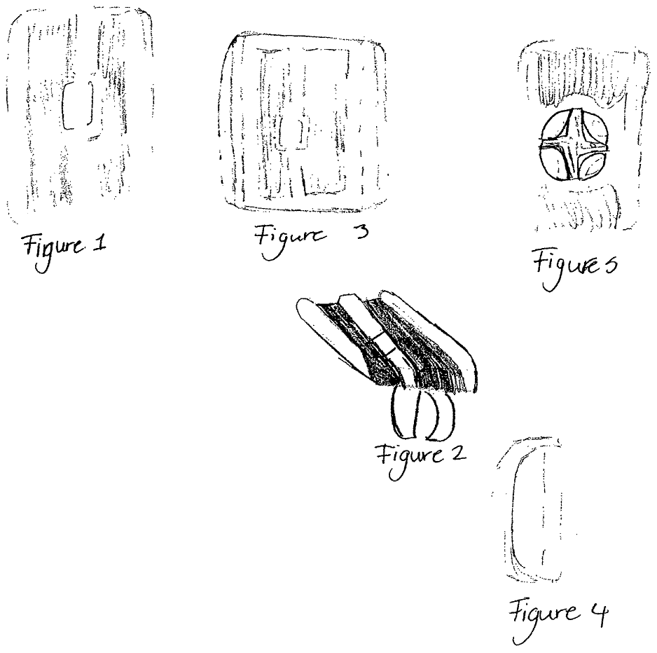

FIG. 1: Front elevation view of the design for a sanitizing clip.

FIG. 2: Right side elevation view thereof: the left side is a mirror image of the right side.

FIG. 3: Rear elevation view thereof.

FIG. 4: Top plain view thereof: the bottom plan is a mirror image of the right side.

FIG. 5: Front, top, and right side perspective view thereof.

1. We claim the user function design the sanitizer clip, as shown and described:
